(19) United States
(12) Patent Application Publication
(10) Pub. No.: US 2025/0032015 A1
(43) Pub. Date: Jan. 30, 2025

(54) BLOOD PHYSIOLOGICAL PARAMETER SENSING DEVICE AND SYSTEM

(71) Applicants: National Taiwan University, Taipei City (TW); National Taiwan University Hospital Hsin-Chu Branch, Zhubei City (TW); National Yang Ming Chiao Tung University, Hsinchu City (TW)

(72) Inventors: Yih-Sharng CHEN, Taipei City (TW); Hsiao-En TSAI, Zhubei City (TW); Yu-Ting CHENG, Hsinchu City (TW)

(73) Assignees: National Taiwan University, Taipei City (TW); National Taiwan University Hospital Hsin-Chu Branch, Zhubei City (TW); National Yang Ming Chiao Tung University, Hsinchu City (TW)

(21) Appl. No.: 18/779,780

(22) Filed: Jul. 22, 2024

Related U.S. Application Data
(60) Provisional application No. 63/528,663, filed on Jul. 25, 2023.

(51) Int. Cl. A61B 5/1468; A61B 5/00

(52) U.S. Cl. CPC A61B 5/1468; A61B 5/6866; A61B 5/742; A61B 2562/227

(57) ABSTRACT

A blood physiological parameter sensing device includes a housing, a flow channel structure, a control unit and a sensing unit. The flow channel structure is disposed in the housing and forms a channel for a liquid to be measured to pass. The control unit is disposed in the housing. The sensing unit is disposed in the housing, is electrically connected to the control unit, and has a sensing end. The sensing end passes through the flow channel structure, is configured in the channel, and has an electrochemical sensing material. The electrochemical sensing material is used to exchange electrons with the blood physiological parameter in the liquid to be measured to produce redox reactions to generate current or voltage change parameters. The sensing unit transmits the current or voltage change parameters to the control unit, which obtains a blood physiological parameter concentration value to achieve instantaneous and continuous monitor the patients.

### CROSS-REFERENCE TO RELATED APPLICATION

This application claims the priority of US provisional application U.S. 63/528,663, filed on Jul. 25, 2023, which is incorporated herewith by reference.

### BACKGROUND OF THE INVENTION

### 1. Field of the Invention

The present invention relates generally to a blood physiological parameter sensing device and system, and more particularly, to a blood physiological parameter sensing device and system capable of instantaneously and continuously sensing blood physiological parameter concentration values in the flowing blood.

### 2. The Prior Arts

Shock patients or patients with tissue ischemic diseases are considered as urgent conditions in acute and critical care and must be closely monitored and treated quickly. Lactate is an indicator of ischemic status. Lactate comes from mitochondria in the cells due to the accumulation of lactate during the production of adenosine triphosphate (ATP) by the anaerobic respiratory pathway. Lactate is a metabolic product of muscle contraction and consumption of carbohydrates. When energy consumption exceeds oxygen supply, the body will undergo a biochemical metabolic pathway of hypoxic anaerobic respiration to produce lactate. Under conditions such as alcoholism, diabetes, hepatic coma, elevated body temperature, malignant tumors, shock, strenuous exercise or hypoxia, the blood lactate concentration will increase; hypothermia will cause the blood lactate concentration to decrease.

According to current research, the higher the blood lactate concentration value, the higher the mortality rate. Therefore, it is necessary in the intensive care unit to frequently monitor the blood lactate concentration in patients with severe shock or emergency care to adjust treatment strategies immediately. The general approach is to directly draw blood from the patient at breakpoints, and analyze the drawn blood using enzymes to obtain the blood lactate concentration value.

Moreover, different diseases require analysis of different blood physiological parameters, such as pH, blood urea nitrogen, blood sugar, cardiac enzymes or various electrolytes. The aforementioned blood physiological parameters are also collected from patients at breakpoints, and the drawn blood is analyzed using enzymes to obtain blood physiological parameters such as pH, blood urea nitrogen, blood sugar, cardiac enzymes, or various electrolytes.

However, the approach of frequent blood drawing at breakpoints will cause the following problems: first, it cannot continuously and immediately reflect blood physiological parameter concentration values; second, it increases the workload of the nursing care team; third, repeated blood drawing causes the patient's pain, iatrogenic anemia, and risk of bloodstream infections.

### SUMMARY OF THE INVENTION

A primary objective of the present invention is to provide a blood physiological parameter sensing device and system that can instantly and continuously sense the blood physiological parameter concentration values in the flowing blood.

For achieving the foregoing objective, the present invention provides a blood physiological parameter sensing device, which includes a housing; a channel structure, disposed inside the housing and having a channel for a liquid to be measured to pass through; a control unit, disposed inside the housing; and a sensing unit, disposed inside the housing, electrically connected to the control unit, and having a sensing end, the sensing end passing through the channel structure, disposed inside the channel, and having an electrochemical sensing material, the electrochemical sensing material being for exchanging electrons with the blood physiological parameters in the liquid to be measured to produce a redox reaction to generate a current or voltage change parameter, and the sensing unit transmitting the current or voltage change parameter to the control unit to obtain a blood physiological parameter concentration value according to the current or voltage change parameter.

In some embodiments, the channel structure includes a body, a first connector and a second connector; the first connector and the second connector are respectively disposed at two ends of the body, the body, the first connector and the second connector jointly form the channel, and the sensing end passes through the body.

In some embodiments, the housing includes a bottom, a first side cover, a second side cover, a base, an upper cover, and a battery, the first side cover and the second side cover are disposed on the base and form a cavity; the body, the control unit, the base, the upper cover, the battery are disposed in the cavity; the control unit is disposed on the bottom, and the base is disposed above the control unit, the body is disposed on the base, the upper cover is disposed on the body, the battery is disposed on the control unit and is electrically connected to the control unit, the first connector passes through the first side cover, and the second connector passes through the second side cover.

In some embodiments, the first side cover has a switch and a USB slot, and the switch and the USB slot are electrically connected to the control unit.

In some embodiments, a groove is opened on the top of the body, and the blood physiological parameter sensing device further includes two waterproof gaskets, and the waterproof gaskets are disposed in the groove and clamp the sensing end.

In order to achieve the aforementioned objective, the present invention provides a blood physiological parameter sensing system, comprising an extracorporeal circulation device, further comprising a host, an input tube and an output tube, the input tube being connected to the host, and the output tube being connected to the host; a display device; and a blood physiological parameter sensing device, further comprising a housing, a channel structure, a control unit, and a sensing unit, the channel structure being disposed inside the housing and comprising a channel, two ends of the channel structure being respectively connected to the input tube and the output tube; the control unit being disposed inside the housing and is electrically connected to the display device or connected with the display device, the sensing unit being disposed inside the housing and electrically connected to the control unit, and having a sensing end, the sensing end passing through the channel structure, being disposed inside the channel, and having an electrochemical sensing material; wherein when the host provides a liquid to be measured, the liquid to be measured sequentially passes through the input tube, the channel and the output tube and returns to the inside of the host to form a circulation loop, and the electrochemical sensing material and the blood physiological parameters in the liquid to be measured produce electron exchange in the redox reaction to generate a current or voltage change parameter; the sensing unit transmits the current or voltage change parameter to the control unit, the control unit obtains a blood physiological parameter concentration value according to the current or voltage change parameter, and the blood physiological parameter concentration value is sent to the display device, and the display device displays the blood physiological parameter concentration value.

In some embodiments, the channel structure includes a body, a first connector and a second connector; the first connector and the second connector are respectively disposed at two ends of the body, the body, the first connector and the second connector jointly form the channel, the sensing end passes through the body; the input tube is connected to the first connector, and the output tube is connected to the second connector.

In some embodiments, the housing includes a bottom, a first side cover, a second side cover, a base, an upper cover, and a battery, the first side cover and the second side cover are disposed on the base and form a cavity; the body, the control unit, the base, the upper cover, the battery are disposed in the cavity; the control unit is disposed on the bottom, and the base is disposed above the control unit, the body is disposed on the base, the upper cover is disposed on the body, the battery is disposed on the control unit and is electrically connected to the control unit, the first connector passes through the first side cover, and the second connector passes through the second side cover.

In some embodiments, the first side cover has a switch and a USB slot, and the switch and the USB slot are electrically connected to the control unit.

In some embodiments, a groove is opened on the top of the body, and the blood physiological parameter sensing device further includes two waterproof gaskets, and the waterproof gaskets are disposed in the groove and clamp the sensing end.

In summary, the present invention can instantly and continuously monitor the blood physiological parameter concentration value in the flowing blood (i.e., the liquid to be measured), and achieve immediate and continuous monitoring of the blood and blood physiology of shock patients or patients with tissue ischemia. The changes in parameter concentration values effectively provide the nursing team with more adequate clinical information. With the present invention, there is no need to draw blood repeatedly throughout the process, which causes patient pain or iatrogenic anemia, and can avoid the risk of blood infection.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a perspective view of the blood physiological parameter sensing system of the present invention;

FIG. 2 is a perspective view of the blood physiological parameter sensing device of the present invention;

FIG. 3 is an exploded view of the blood physiological parameter sensing device of the present invention;

FIG. 4 is a cross-sectional view taken along line IV-IV in FIG. 2;

FIG. 5 is a cross-sectional view taken along line V-V in FIG. 2;

FIG. 6 is a perspective view of the sensing unit of the present invention;

FIG. 7 is a schematic view of the flow of liquid to be measured in the blood physiological parameter sensing system of the present invention;

FIG. 8 is a schematic view of the liquid to be measured passing through the channel structure of the present invention; and

FIG. 9 is a schematic view of signal transmission of the sensing unit, the control unit, and the display device of the present invention

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

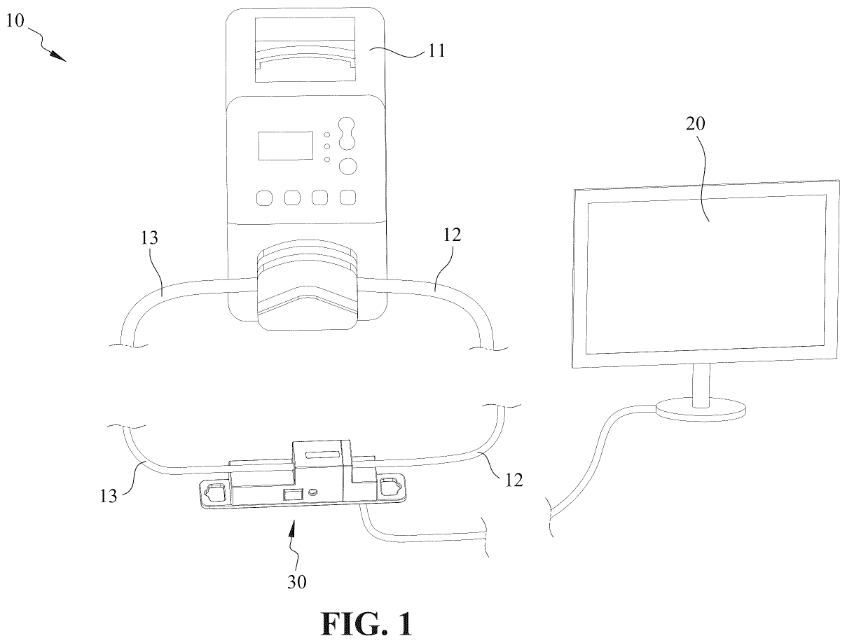

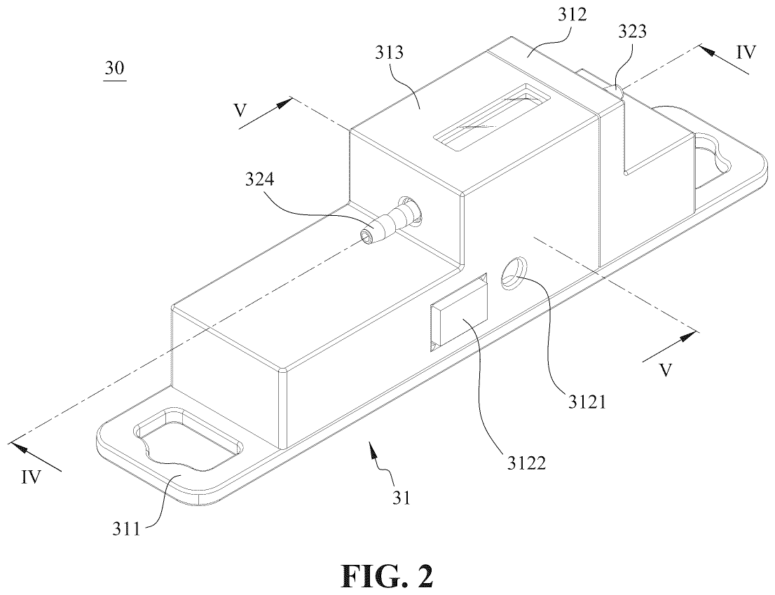

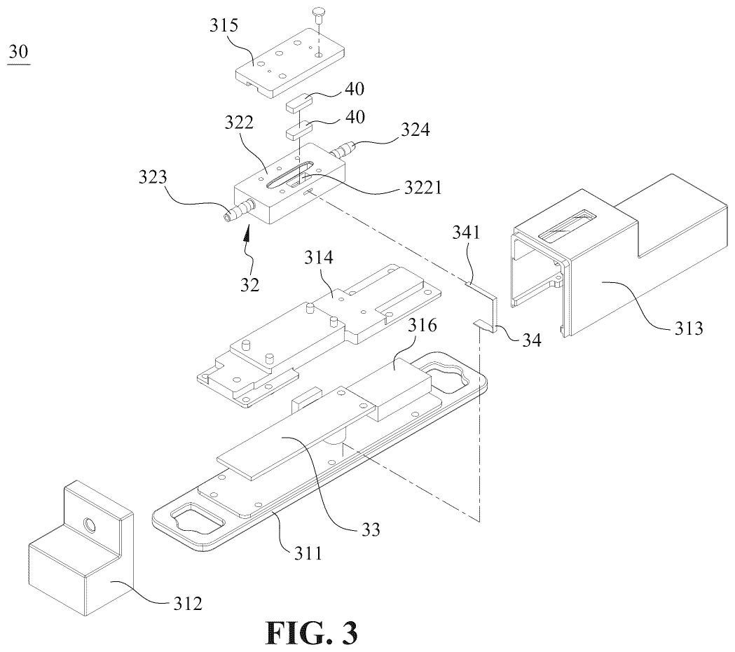

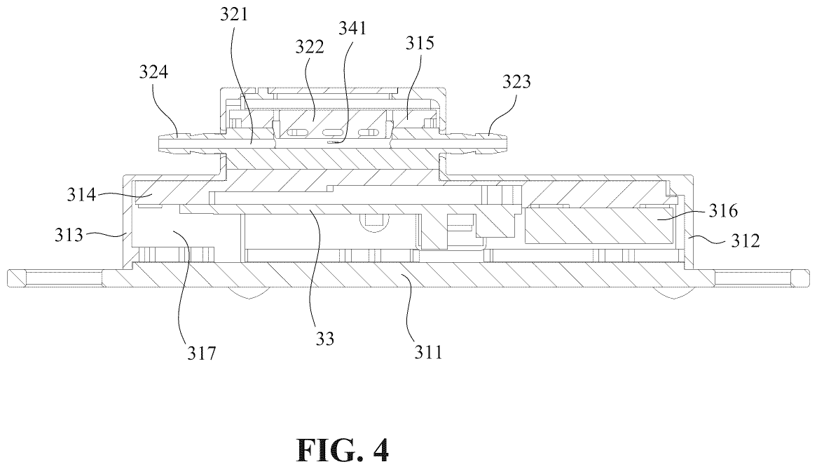

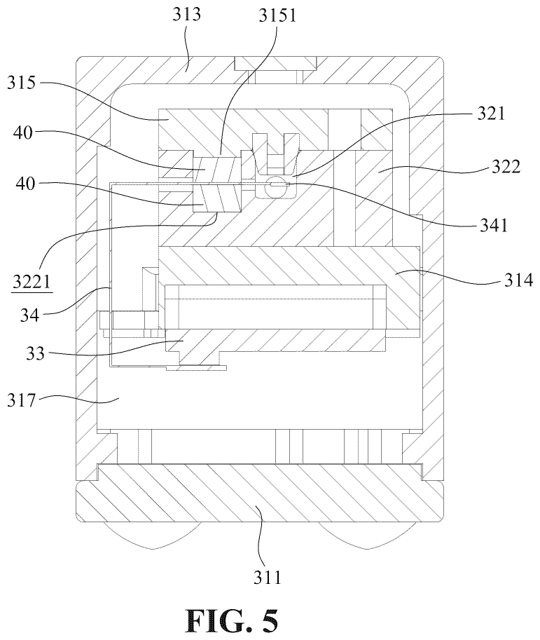

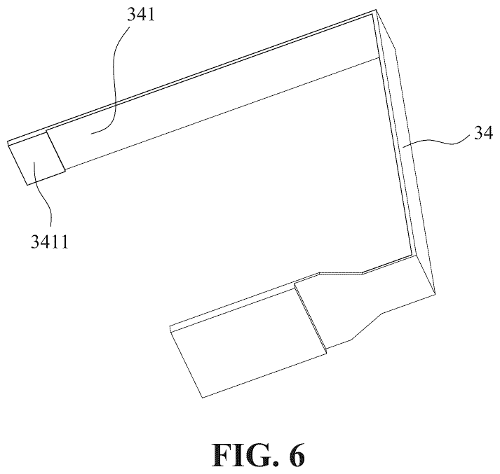

FIG. 1 is a perspective view of the blood physiological parameter sensing system of the present invention. FIG. 2 is a perspective view of the blood physiological parameter sensing device 30 of the present invention. FIG. 3 is an exploded view of the blood physiological parameter sensing device 30 of the present invention. FIG. 4 is a cross-sectional view taken along line IV-IV in FIG. 2. FIG. 5 is a cross-sectional view taken along line V-V in FIG. 2. FIG. 6 is a perspective view of the sensing unit of the present invention. The present invention provides a blood physiological parameter sensing system, which includes an extracorporeal circulation device 10, a display device 20, and a blood physiological parameter sensing device 30.

As shown in FIG. 1, the extracorporeal circulation device 10 includes a host 11, an input tube 12 and an output tube 13. The input tube 12 is connected to the host 11, and the output tube 13 is connected to the host 11. Preferably, the host 11 can be an ECMO membrane device, an extracorporeal cardiopulmonary circulation device or a hemodialysis pipeline, thereby providing the patient's flowing blood (i.e., the liquid to be measured), so that the blood physiological parameter sensing system of the present invention can be applied to a patient that requires close monitoring of changes in blood physiological parameter concentrations.

As shown in FIG. 1, the display device 20 can be a display screen of an electronic device such as a mobile phone, tablet computer, notebook computer or desktop computer. The user can monitor the blood physiological parameter concentration values continuously and in real time through the screen of mobile phone, tablet computer, notebook computer, desktop computer, or other electronic devices.

As shown in FIGS. 1 to 6, the blood physiological parameter sensing device 30 includes a housing 31, a channel structure 32, a control unit 33, and a sensing unit 34. The channel structure 32 is disposed inside the housing 31 and defines a channel 321. Two ends of the channel structure 32 are connected to the input tube 12 and the output tube 13 respectively. The control unit 33 is disposed inside the housing 31 and is electrically connected to or wired with the display device 20. The sensing unit 34 is disposed inside the housing 31, is electrically connected to the control unit 33, and has a sensing end 341. The sensing end 341 passes through the channel structure 32, is disposed inside the channel 321, and has an electrochemical sensing material 3411.

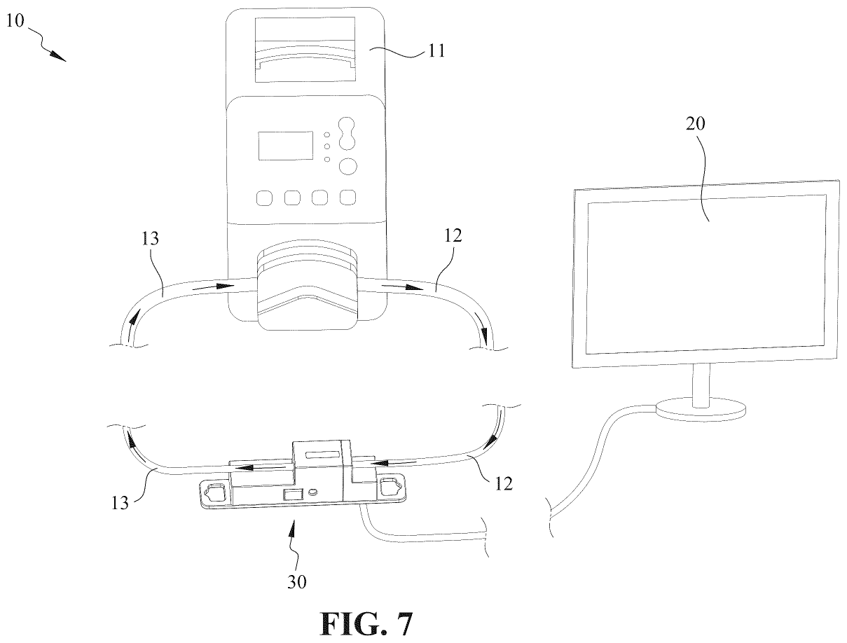

FIG. 7 is a schematic view of the flow of liquid to be measured in the blood physiological parameter sensing system of the present invention. As shown in FIG. 7, when the host 11 provides a liquid to be measured (flowing blood), the liquid to be measured sequentially passes through the input tube 12, the channel 321, and the output tube 13 and returns to the inside of the host 11 to form a circulation loop.

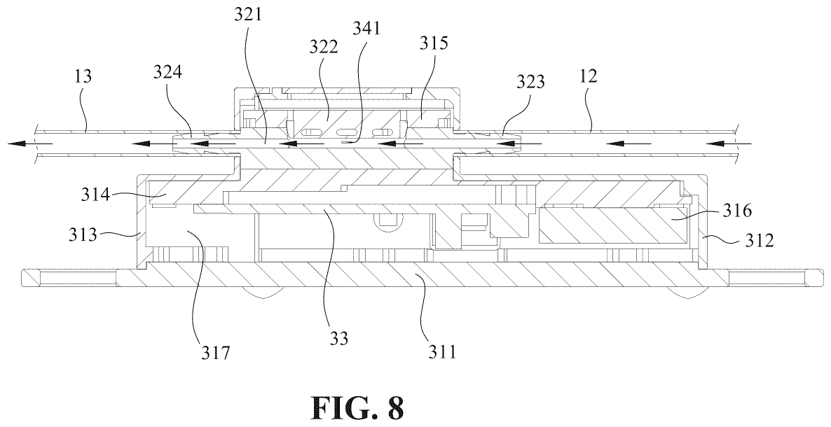

FIG. 8 is a schematic view of the liquid to be measured passing through the channel structure 32 of the present invention. As shown in FIG. 8, the electrochemical sensing material 3411 induces an electron exchange of a redox reaction with the blood physiological parameter in the liquid to be measured to generate a current or voltage change parameter.

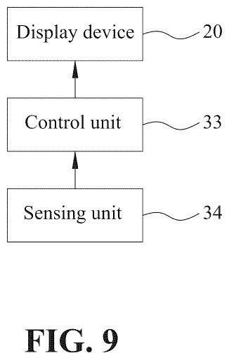

FIG. 9 is a schematic view of signal transmission of the sensing unit 34, the control unit 33, and the display device 20 of the present invention. As shown in FIG. 9, the sensing unit 34 transmits the current or voltage change parameter to the control unit 33. The control unit 33 obtains a blood physiological parameter concentration value according to the current or voltage change parameter and transmits the blood physiological parameter concentration value to the display device 20, and the display device 20 displays the blood physiological parameter concentration value.

As such, the present invention can instantly (i.e., in real-time) and continuously monitor the blood physiological parameter concentration value in the flowing blood (i.e., the liquid to be measured), and achieve real-time and continuous monitoring of the blood physiological parameter concentration value change of the shock patients or patient tissue ischemia. This real-time change information effectively provides the nursing team with more adequate clinical information, eliminates the need for repeated blood draws throughout the process, does not cause patient pain and iatrogenic anemia, and reduces the risk of blood infection.

The blood physiological parameters may include lactate, pH, blood urea nitrogen, blood sugar, cardiac enzymes or various electrolytes, etc. However, the present invention is not limited thereto, and any blood physiological parameters can be monitored using the present invention.

Preferably, the sensing unit 34 is a flexible printed circuit board, which is light in weight, thin in thickness, soft, and bendable. Nanoscale electrochemical sensing materials 3411, such as metal oxides or ionic liquids, are sprayed on the sensing end 341. Furthermore, the known enzyme analysis method is limited to a single measurement and cannot provide instant and continuous measurement. Therefore, the present invention can achieve the effect of rapid, real-time, and continuous monitoring of the blood physiological parameter concentration value of the liquid to be measured through electrochemical and non-enzyme sensing principles.

As shown in FIGS. 2, 3 and 4, in a preferred embodiment, the channel structure 32 includes a body 322, a first connector 323, and a second connector 324, are the first connector 323 and the second connector 324 are respectively disposed at two ends of the body 322. The body 322, the first connector 323, and the second connector 324 together form a channel 321. The sensing end 341 passes through the body 322. The input tube 12 is connected to the first connector 323, and the output tube 13 is connected to second connector 324.

As shown in FIGS. 3 and 5, in a preferred embodiment, a groove 3221 is provided on the top of the body 322, and the blood physiological parameter sensing device 30 further includes two waterproof gaskets 40. These waterproof gaskets 40 are disposed in the groove 3221 and clamp the sensing end 341. Thereby, the waterproof gaskets 40 can fix the sensing end 341 and prevent the liquid to be measured from leaking. Preferably, the waterproof gaskets 40 are made of silicone, however, the present invention is not limited thereto.

As shown in FIGS. 2 and 3, in the preferred embodiment, the housing 31 includes a bottom 311, a first side cover 312, a second side cover 313, a base 314, an upper cover 315, and a battery 316. The first side cover 312 and the second side cover 313 are disposed on the bottom 311 and form a cavity 317. The body 322, the control unit 33, the base 314, the upper cover 315, and the battery 316 are all disposed inside the cavity 317. The control unit 33 is disposed on the bottom 311, the base 314 is arranged above the control unit 33, the body 322 is arranged on the base 314, the upper cover 315 is arranged on the body 322, the battery 316 is arranged on the control unit 33 and is electrically connected to the control unit 33. The first connector 323 passes through the first side cover 312, and the second connector 324 passes through the second side cover 313.

As shown in FIG. 2, in the preferred embodiment, the first side cover 312 has a switch 3121 and a USB slot 3122. The switch 3121 and the USB slot 3122 are electrically connected to the control unit 33. The user can turn on and off the blood physiological parameter sensing device 30 through the switch 3121. The user can plug the USB into the USB slot 3122 to obtain blood physiological parameter concentration values.

As shown in FIG. 5, in a preferred embodiment, the upper cover 315 has a protrusion 3151. The protrusion 3151 is disposed in the groove 3221 and presses on one of the waterproof gaskets 40 to prevent one of the waterproof gaskets 40 from dislodging out of the groove 3221.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A blood physiological parameter sensing device, comprising:
a housing;
a channel structure, disposed inside the housing and having a channel for a liquid to be measured to pass through;
a control unit, disposed inside the housing; and
a sensing unit, disposed inside the housing, electrically connected to the control unit, and having a sensing end, the sensing end passing through the channel structure, disposed inside the channel, and having an electrochemical sensing material, the electrochemical sensing material being for exchanging electrons with the blood physiological parameters in the liquid to be measured to produce a redox reaction to generate a current or voltage change parameter, and the sensing unit transmitting the current or voltage change parameter to the control unit to obtain a blood physiological parameter concentration value according to the current or voltage change parameter.

2. The blood physiological parameter sensing device according to claim 1, wherein the channel structure comprises a body, a first connector and a second connector; the first connector and the second connector are respectively disposed at two ends of the body, the body, the first connector and the second connector jointly form the channel, and the sensing end passes through the body.

3. The blood physiological parameter sensing device according to claim 2, wherein the housing comprises a bottom, a first side cover, a second side cover, a base, an upper cover, and a battery, the first side cover and the second side cover are disposed on the base and form a cavity; the body, the control unit, the base, the upper cover, the battery are disposed in the cavity; the control unit is disposed on the bottom, and the base is disposed above the control unit, the body is disposed on the base, the upper cover is disposed on the body, the battery is disposed on the control unit and is electrically connected to the control unit, the first connector passes through the first side cover, and the second connector passes through the second side cover.

4. The blood physiological parameter sensing device according to claim 3, wherein a groove is opened on the top of the body, and the blood physiological parameter sensing device further includes two waterproof gaskets, and the waterproof gaskets are disposed in the groove and clamp the sensing end.

5. The blood physiological parameter sensing device according to claim 2, wherein the first side cover has a switch and a USB slot, and the switch and the USB slot are electrically connected to the control unit.

6. A blood physiological parameter sensing system, comprising:
an extracorporeal circulation device, further comprising a host, an input tube, and an output tube, the input tube being connected to the host, and the output tube being connected to the host;
a display device; and
a blood physiological parameter sensing device, further comprising a housing, a channel structure, a control unit, and a sensing unit, the channel structure being disposed inside the housing and comprising a channel, two ends of the channel structure being respectively connected to the input tube and the output tube; the control unit being disposed inside the housing and is electrically connected to the display device or connected with the display device, the sensing unit being disposed inside the housing and electrically connected to the control unit, and having a sensing end, the sensing end passing through the channel structure, being disposed inside the channel, and having an electrochemical sensing material;
wherein when the host provides a liquid to be measured, the liquid to be measured sequentially passes through the input tube, the channel and the output tube and returns to the inside of the host to form a circulation loop, and the electrochemical sensing material and the blood physiological parameters in the liquid to be measured produce electron exchange in the redox reaction to generate a current or voltage change parameter; the sensing unit transmits the current or voltage change parameter to the control unit, the control unit obtains a blood physiological parameter concentration value according to the current or voltage change parameter, and the blood physiological parameter concentration value is sent to the display device, and the display device displays the blood physiological parameter concentration value.

7. The blood physiological parameter sensing system according to claim 6, wherein the channel structure comprises a body, a first connector and a second connector; the first connector and the second connector are respectively disposed at two ends of the body, the body, the first connector and the second connector jointly form the channel, and the sensing end passes through the body; the input tube is connected to the first connector, and the output tube is connected to the second connector.

8. The blood physiological parameter sensing system according to claim 7, wherein the housing comprises a bottom, a first side cover, a second side cover, a base, an upper cover, and a battery, the first side cover and the second side cover are disposed on the base and form a cavity; the body, the control unit, the base, the upper cover, the battery are disposed in the cavity; the control unit is disposed on the bottom, and the base is disposed above the control unit, the body is disposed on the base, the upper cover is disposed on the body, the battery is disposed on the control unit and is electrically connected to the control unit, the first connector passes through the first side cover, and the second connector passes through the second side cover.

9. The blood physiological parameter sensing system according to claim 8, wherein a groove is opened on the top of the body, and the blood physiological parameter sensing device further includes two waterproof gaskets, and the waterproof gaskets are disposed in the groove and clamp the sensing end.

10. The blood physiological parameter sensing system according to claim 7, wherein the first side cover has a switch and a USB slot, and the switch and the USB slot are electrically connected to the control unit.
